(19) United States
(12) Patent Application Publication
(10) Pub. No.: US 2006/0058640 A1
(43) Pub. Date: Mar. 16, 2006

(54) System for positioning an observation and/or intervention device on a patient

(76) Inventors: Philippe Cinquin, Saint Nazaire Les Eymes (FR); Alain Jacquet, Moretel De Mailles (FR); Elise Taillant, Grenoble (FR)

Correspondence Address: PLEVY & HOWARD, P.C., P.O. BOX 226, FORT WASHINGTON, PA, 19034, US

(21) Appl. No.: 11/224,722

(22) Filed: Sep. 12, 2005

(30) Foreign Application Priority Data
Sep. 13, 2004 (FR) 04/52023

(51) Int. Cl. A61B 5/05

(52) U.S. Cl. 600/415

(57) ABSTRACT

A system for positioning with respect to a patient's body an observation and/or intervention device comprising at least a mechanical part and a system for actuating the mechanical part, the actuation system comprising a piston capable of sliding a rod and connected to means for supplying a fluid under pressure; a drive element connected to the mechanical part and comprising dissymmetrical teeth; and a bearing element assembled on the rod and capable, on displacement of the rod along a determined direction, of cooperating with a tooth of the drive element to displace the drive element by a determined distance and/or angle, the bearing element, on displacement of the rod along the direction opposite to the determined direction, exerting a substantially zero force on the drive element to avoid displacing the drive element.

### BACKGROUND OF THE INVENTION

1. Field of the Invention

The present invention relates to a system for positioning on a patient an observation and/or intervention device, for example, an observation and/or intervention device having a portion penetrating into the patient's body. It for example applies to medical analysis devices, such as needles, and it will be more specifically described in the case of the use of a hollow needle in a biopsy.

2. Discussion of the Related Art

In such a type of operation, a hollow needle formed of a thin tube is inserted, for example, in the patient's abdomen, thorax, or back through a small incision performed at the patient's level. The operation is generally carried out with the assistance of images obtained by a medical imaging method, for example, a scanner imaging or magnetic resonance imaging (MRI) method, to help the radiologist visualize the patient's interior.

The puncture operation then generally comprises the alternation of image acquisition phases and of phases where the actual puncture is performed. The obtained images are cross-section views enabling the radiologist to determine whether the needle orientation is or not correct, and, possibly, to correct this orientation. Such a transcription of the needle position in the three-dimensional space from the position of the needle on two-dimensional images is difficult and requires much experience from the radiologist for the intervention to be accurately performed.

Further, if the used imaging system is a scanner and if the radiologist must remain in the room where the patient is, for example, to maintain the needle during the image acquisition, he is then submitted to the radiations emanating from the imaging system, which may be dangerous, given the number of interventions performed by the radiologist along the year.

Further, when the used imaging system is an MRI system, it is necessary to use a so-called “open” MRI system, comprising a tunnel which is not completely closed to enable access to the patient. Such systems provide images having a lesser quality than that obtained with so-called “closed” MRI systems.

To overcome the previously-described disadvantages, robotic systems have thus been developed to handle the needle instead of the radiologist.

As an example, French application 02/05848 filed by PRAXIM Company describes a system for positioning on a patient an observation and/or intervention device taking up a small volume. The displacement and the orientation of the positioning system are then generally performed based on the analysis of the images obtained by the used imaging system. The positioning system is displaced and oriented via electric actuators. The electric actuators may be arranged at the positioning system level and then directly drive mechanical parts of the positioning system, possibly by gear reduction and transmission systems. The electric actuators may be arranged at a distance from the positioning system, each electric actuator then driving a cable connected to a mechanical part of the positioning system.

A disadvantage of such a positioning system is that it is generally difficult to make it compatible with the sterility constraints specific to a medical operation. Indeed, it is then necessary to provide a method for sterilizing the positioning system, but also electric actuators and/or cables connecting the electric actuators to the positioning system, which may turn out to be difficult and/or expensive.

Another disadvantage is that electric actuators, or the cables connecting the electric actuators to the positioning system, in addition to leaving marks on an image obtained by currently-used medical imaging methods, may deform the rest of the image, which may hinder the analysis thereof.

### SUMMARY OF THE INVENTION

The present invention aims at obtaining a system for positioning on a patient an observation and/or intervention device and which is actuated via actuators that may easily be made compatible with the sterility constraints specific to a medical operation.

According to another object, the present invention provides a positioning system actuated by actuators that may, if necessary, be formed of materials leaving little or no marks on images obtained by medical imaging methods of scanner or MRI type or at least, if they leave marks on such images, that do not deform the rest of the images.

To achieve these objects, the present invention provides a system for positioning with respect to a patient's body an observation and/or intervention device comprising at least a mechanical part and a system for actuating the mechanical part. The actuation system comprises a piston capable of sliding a rod and connected to means for supplying a fluid under pressure; a drive element connected to the mechanical part and comprising dissymmetrical teeth; and a bearing element assembled on the rod and capable, on displacement of the rod along a determined direction, of cooperating with a tooth of the drive element to displace the drive element by a determined distance and/or angle, the bearing element, on displacement of the rod along the direction opposite to the determined direction, exerting a substantially zero force on the drive element to avoid displacing the drive element.

According to an object of the present invention, the drive element is a ratchet wheel.

According to an object of the present invention, the fluid is air.

According to an object of the present invention, the actuation system is totally formed of materials leaving no marks on an image obtained by a scanner imaging or magnetic resonance imaging system or leaving marks on said image without deforming the rest of said image.

According to an object of the present invention, the positioning system comprises a drive system comprising a shaft and first and second actuation systems, the first actuation system being capable of rotating the shaft along a determined rotation direction and the second actuation system being capable of rotating the shaft along the direction opposite to the determined rotation direction.

According to an object of the present invention, the positioning system comprises a base to which said device is connected according to a number of degrees of liberty; flexible connection means, each of which is arranged between the base and a frame solid with the patient's support or the patient himself; and drive means for modifying the length/tension of the connection means.

According to an object of the present invention, the positioning system comprises a base laid on the patient's body; means for supporting the device, formed of at least a first portion movably assembled on the base according to a connection with one degree of liberty, and of a second portion movably assembled on the first portion according to a connection with one degree of liberty and connected to the device; and a first system for driving the first portion with respect to the base, and a second system for driving the second portion with respect to the first portion.

According to an object of the present invention, the first portion is a mobile ring comprising a toothed wheel having an axis substantially perpendicular to the patient's body, the first drive system comprising an endless screw fixedly attached to the shaft and capable of cooperating with the toothed wheel to rotate the toothed wheel around its axis.

According to an object of the present invention, the second portion is pivotally assembled on the mobile ring along an axis substantially tangent to the patient's body, a toothed wheel sector being attached to the mobile ring, the second portion comprising an endless screw cooperating with the toothed wheel sector, the shaft of the second drive system rotating the endless screw.

According to an object of the present invention, the means for supporting the device comprise a third portion slidably assembled on the second portion and connected to the device, the system comprising a third system for driving the third portion with respect to the second portion.

The foregoing objects, features, and advantages of the present invention, as well as others, will be discussed in detail in the following non-limiting description of a specific example of embodiment in connection with the accompanying drawings.

### BRIEF DESCRIPTION OF THE DRAWINGS

FIG. 1 is a simplified top view of an example of the forming of a positioning system according to the present invention;

FIG. 2 is a perspective view of a portion of the positioning system of FIG. 1;

FIG. 3 is a simplified view illustrating the operation of an actuator of the positioning system according to the present invention;

FIG. 4 is a cut-away view of a portion of FIG. 1; and

FIG. 5 is a cut-away view of a portion of FIG. 1.

### DETAILED DESCRIPTION

For clarity, same elements have been designated with same reference numerals in the different drawings.

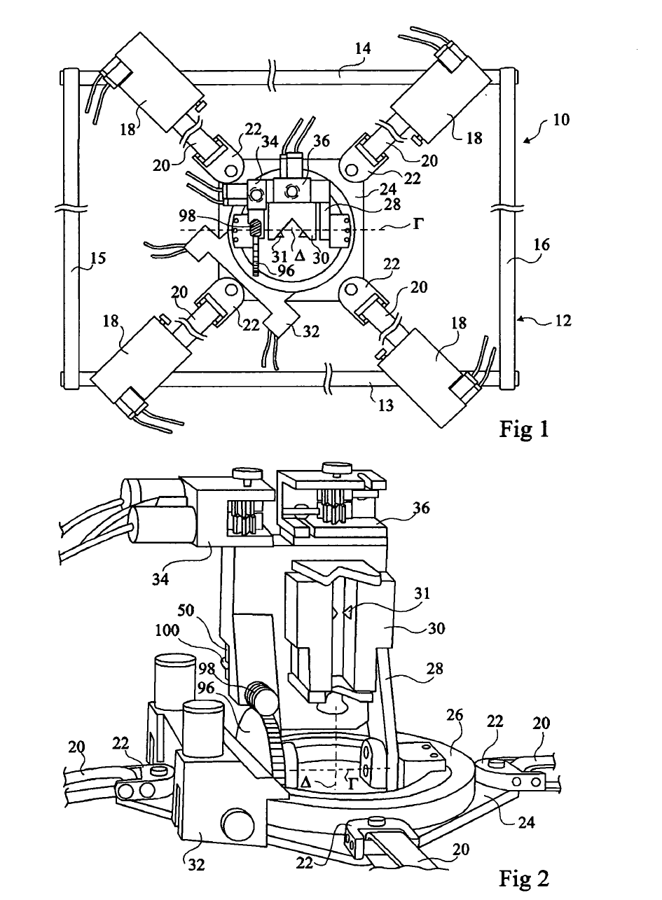

FIGS. 1 and 2 show an example of the forming of positioning system 10 according to the present invention. Positioning system 10 comprises a frame 12, formed of two lateral members 13, 14, an upper member 15, and a lower member 16. Positioning system 10 is intended, for example, to be placed at the level of the abdomen, the thorax, or the back of a patient who is, for example, lying on an operating table. Lateral walls 13, 14 of frame 12 are then attached to the operating table, upper and lower walls 15, 16 overstriding the patient's abdomen, thorax, or back.

Two drive systems 18 are attached on each lateral members 13, 14. The drive systems are attached, for example, substantially symmetrically with respect to the center of frame 12. Each drive system 18 is capable of exerting a stronger or lighter traction on a flexible strip 20 having an end connected to a loop 22. The four loops 22 are pivotally assembled at the four ends of a substantially rectangular base 24 and laid on the patient's abdomen, thorax, or back. The control of drive systems 18 enables exerting different tractions on flexible strips 20, causing the displacement of base 24 on the patient's abdomen, thorax, or back.

Base 24 supports a mobile ring 26 of axis Δ substantially perpendicular to the plane tangent to the abdomen, to the thorax, or to the back at the level where the incision must be performed. Mobile ring 26 is rotatably assembled around axis Δ on base 24 via a bearing, not shown.

A stirrup 28 is pivotally assembled on mobile ring 26 along an axis Γ substantially perpendicular to axis Δ, and substantially comprised in the plane tangent to the abdomen, to the thorax, or to the back at the incision level. A head 30 is slidably assembled on stirrup 28. Head 30 comprises pliers 31 intended to receive an observation and/or intervention device (not shown), for example, a puncture needle penetrating into the patient through a small incision. Head 30 may be displaced with respect to stirrup 28 to be brought closer or moved away from the patient. A drive system 32 is attached to base 24 and enables driving mobile ring 26 around axis Δ with respect to base 24. A drive system 34 is attached to stirrup 28 and enables pivoting stirrup 28 with respect to mobile ring 26 around axis Γ. A drive system 36 is attached to stirrup 28 and enables causing the displacement of head 30 with respect to stirrup 28 to bring it closer or move it away from the patient. Positioning system 10 may comprise a drive system, not shown, capable of actuating pliers 31 or head 30. All drive systems 18, 32, 34, and 36 according to the present invention have a similar structure.

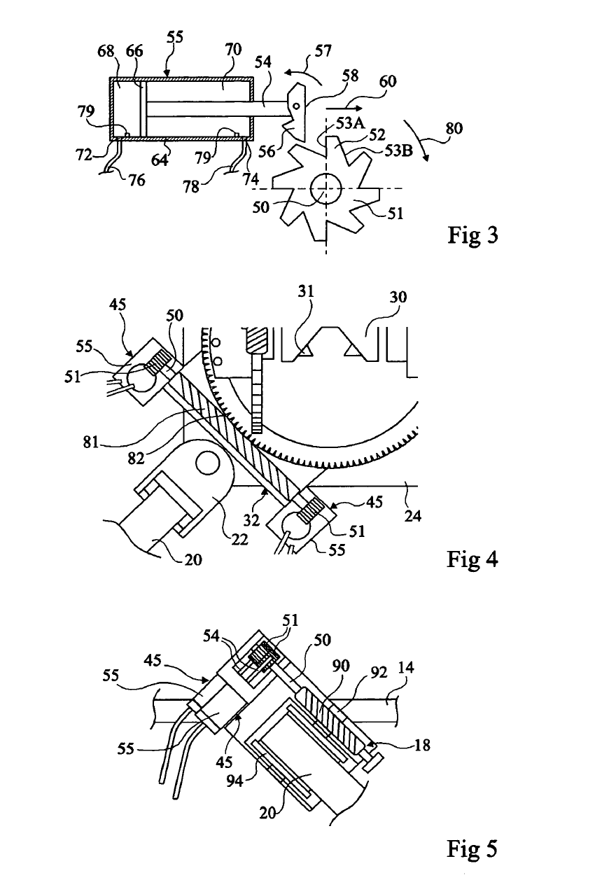

FIG. 3 illustrates the operating principle of an actuation system 45 which forms the base element of each drive system 18, 32, 34, and 36 of the positioning system according to the present invention.

Actuation system 45 comprises an output shaft 50 on which is assembled a ratchet wheel 51. Ratchet wheel 51 comprises dissymmetrical teeth 52, each comprising a first face 53A and a second face 53B. First face 53A is comprised within a plane containing the axis of wheel 51 and second face 53B is comprised within a plane inclined with respect to the plane of the corresponding first face 53A. The rotating of ratchet wheel 51 is performed by rod 54 of a piston 55. Rod 54 supports at one end a bearing element 56 assembled on the end of rod 54 around an axis parallel to the axis of shaft 50, to be able to pivot with respect to rod 54 from an idle position in the rotation direction indicated by arrow 57. From the idle position shown in FIG. 3, bearing element 56 cannot pivot with respect to rod 54 in the rotation direction opposite to arrow 57. Bearing element 56 comprises a bearing surface 58 capable of cooperating with first face 53A and second face 53B of dissymmetrical teeth 52 of ratchet wheel 51 on displacement of rod 54. Piston 55 is capable of sliding rod 54 in the direction indicated by arrow 60 or in the opposite direction. Advantageously, piston 55 is a dual-effect air piston comprising an enclosure 64 in which a wall 66 tightly separating enclosure 64 in two chambers 68, 70 can slide. Each chamber 68, 70 may be supplied with air under pressure, for example, compressed air under three bars, through openings 72, 74 connected to feed pipes 76, 78. Stops 79 are provided in each chamber 68, 70 to limit the travel of wall 66 and thus the travel of rod 54.

The operating principle of actuation system 45 is the following. Ratchet wheel 51 is in an idle position such that first face 53A of a tooth 52 is substantially parallel to bearing surface 58. When rod 54 displaces along the direction indicated by arrow 60, which means that air under pressure is sent into chamber 68, bearing surface 58 of bearing element 56 bears against first face 53A of a tooth 52 of wheel 51. Since bearing element 56 then cannot pivot with respect to rod 54, it exerts a thrust on tooth 52, which causes the rotating of wheel 51 in the rotation direction indicated by arrow 80 while head 58 cooperates with tooth 52. At one time, the progress of rod 54 and the rotating of wheel 51 are such that bearing element 56 is no longer in contact with tooth 52, immobilizing wheel 51. Wheel 51 has then generally moved by a determined angle which corresponds to the angle between two planes containing two first faces 53A of two adjacent teeth 52 of wheel 51.

When rod 54 is placed along the direction opposite to arrow 60, which means that air under pressure is sent into chamber 70, bearing element 56 comes into contact with another tooth of wheel 51 that then takes up the initial place where the tooth with which bearing element 56 has come into contact in the previous motion of rod 54 used to be. Bearing element 56 then pivots with respect to rod 54. No action is thus applied by bearing element 56 on ratchet wheel 51. Ratchet wheel 51 is then not rotated in a back motion of rod 54.

FIG. 4 shows a cut-away view of drive system 32. Drive system 32 comprises two actuation systems 45 such as previously described, and which have the same output shaft 50. The actuation systems are arranged at the level of each end of shaft 50. An endless screw 81 is assembled to move along with shaft 50. Actuation systems 45 are arranged so that the driving of a ratchet wheel of one of the actuation systems causes the rotating of shaft 50 in a rotation direction and the driving of the ratchet wheel of the other actuation system causes the rotating of shaft 50 in the opposite rotation direction. Mobile ring 26 comprises a toothed wheel 82 which permanently meshes with endless screw 81. A rotation of endless screw 81 then rotates toothed wheel 82 around axis Δ. A reciprocal motion of the piston 55 of an actuation system 45 causing the rotation of the associated ratchet wheel 51 by a determined angle, this results in a rotation by an also determined angle of toothed wheel 82. The rotation of toothed wheel 82, and thus of mobile ring 26, with respect to base 24 can thus be controlled by the control of the two actuation systems 45 of drive system 32.

FIG. 5 shows a cut-away view of drive system 18. Drive system 18 comprises two actuation systems 45, such as previously described, and which have the same output shaft 50. The actuation systems are arranged at the level of a same end of shaft 50. An endless screw 90 is assembled to move along with shaft 50. Actuation systems 45 are arranged so that the driving of a ratchet wheel 51 of one of the actuation systems causes the rotating of shaft 50 in a rotation direction and the driving of ratchet wheel 51 of the other actuation system causes the rotation of wheel 50 in the opposite rotation direction. Endless screw 90 cooperates with a toothed wheel 92 coaxially assembled with a pulley 94 on which flexible strip 20 is wound. A reciprocal motion of a piston 55 of an actuation system 45 causing the rotation of an associated ratchet wheel 51 by a determined angle, this results in a displacement of a determined length of flexible strip 20 (which depends on the strip length wound around pulley 94). The rotation of toothed wheel 82, and thus the traction of flexible strip 20 can thus be controlled by the control of the two actuation systems 45 of drive system 18.

Drive systems 34, 36 have a structure similar to that of drive system 18. As appears in further detail in FIGS. 1 and 2, positioning system 10 comprises a toothed wheel sector 96 attached to mobile ring 26. An endless screw 98 is rotatably assembled on stirrup 28 while being maintained fixed in translation along its axis with respect to stirrup 28. Endless screw 98 cooperates with toothed wheel sector 96 and is rotated by a transmission gear wheel 100, itself driven by the common output shaft 50 of actuation systems 45 of drive system 34.

Similarly, the displacement of head 30 with respect to stirrup 28 may be obtained by an endless screw (not shown) driven by the common output shaft of actuation systems 45 of drive system 36 and which cooperates with a finger provided at the level of head 30.

The feed pipes of the pistons of the actuation systems of drive systems 18, 32, 34, and 36 are connected to a control package containing valves controlled by means of a computer.

The displacement motion of head 30 with respect to stirrup 28 may be performed in two steps. A fast displacement of head 30 may be obtained by means of an air piston, not shown, having its rod directly driving head 30. This especially enables piercing the skin or capsules of certain organs. A slow displacement of head 30 with respect to stirrup 28 can then be obtained by drive system 36.

According to a variation of the present invention, the pistons associated with the ratchet wheels may be hydraulic pistons.

The present invention enables forming all the components of the previously-described drive systems in materials “compatible” with scanner imaging or MRI methods. These are materials, such as for example plastic materials, which leave no or little marks on images obtained by such imaging methods, or materials, such as for example, titanium, carbon fibers, ceramics, etc., which, although they can leave marks on the obtained images, do not modify the rest of the images. Further, in the case where the fluid used by the pistons of the actuation systems is air, the presence of marks on images obtained by scanner or MRI is further reduced.

Further, since the previously-described drive systems may be formed of materials from among plastic matters, titanium, ceramics, carbon fibers, etc., the replacing or the sterilization thereof may easily be provided on each operation to fulfill the sterilization constraints imposed by the envisaged operation. Further, in the case where the fluid used by the pistons of the actuation systems is air, infection risks are limited in case of a leakage at the piston level.

Of course, the present invention is likely to have various alterations and modifications which will readily occur to those skilled in the art. In particular, in the previously-described example of embodiment, the different motions of the robot are obtained in each case by the driving of a shaft by means of a ratchet wheel, itself actuated by a mobile piston. A rectilinear bar comprising dissymmetrical teeth similar to the teeth of a ratchet wheel may as a variation be provided. The bar displacement would then be performed similarly to what has been previously described via a piston rod. Further, in the previously-described example of embodiment, the pistons are of dual-effect type. Single-acting pistons may of course be provided, the return motion of the piston rod being then obtained by a pull-back system, for example, a spring. Moreover, the flexible strips connected to the base may be replaced with cables or threads.

Such alterations, modifications, and improvements are intended to be part of this disclosure, and are intended to be within the spirit and the scope of the present invention. Accordingly, the foregoing description is by way of example only and is not intended to be limiting. The present invention is limited only as defined in the following claims and the equivalents thereto.

What is claimed is:

1. A system (10) for positioning with respect to a patient's body an observation and/or intervention device comprising at least a mechanical part (20, 26, 28, 30) and a system (45) for actuating the mechanical part, the actuation system comprising:
a piston (55) capable of sliding a rod (54) and connected to means (74, 76) for supplying a fluid under pressure;
a drive element (51) connected to the mechanical part and comprising dissymmetrical teeth (52); and
a bearing element (56) assembled on the rod and capable, on displacement of the rod along a determined direction, of cooperating with a tooth of the drive element to displace the drive element by a determined distance and/or angle, the bearing element, on displacement of the rod along the direction opposite to the determined direction, exerting a substantially zero force on the drive element to avoid displacing the drive element.

2. The system of claim 1, wherein the drive element (51) is a ratchet wheel.

3. The system of claim 1, wherein the fluid is air.

4. The system of claim 1, wherein the actuation system (45) is totally formed of materials leaving no marks on an image obtained by a scanner imaging or magnetic resonance imaging system, or leaving marks on said image without deforming the rest of said image.

5. The system of claim 1, comprising a drive system (18, 32, 34, 36) comprising a shaft (50) and first and second actuation systems (45), the first actuation system being capable of rotating the shaft along a determined rotation direction and the second actuation system being capable of rotating the shaft along the direction opposite to the determined rotation direction.

6. The system of claim 5, comprising:
a base (24) to which said device is connected according to a number of degrees of liberty;
flexible connection means (20), each of which is arranged between the base and a frame (12) solid with the patient's support or the patient himself; and
drive means (18) for modifying the length/tension of the connection means.

7. The system of claim 5, comprising:
a base (24) laid on the patient's body;
means for supporting the device formed of at least a first portion (26) movably assembled on the base according to a connection with one degree of liberty, and of a second portion (30) movably assembled on the first portion according to a connection with one degree of liberty and connected to the device; and
a first system (32) for driving the first portion with respect to the base, and a second system (34) for driving the second portion with respect to the first portion.

8. The system of claim 7, wherein the first portion (26) is a mobile ring comprising a toothed wheel (82) having an axis (Δ) substantially perpendicular to the patient's body, the first drive system (32) comprising an endless screw (81) fixedly attached to the shaft (50) and capable of cooperating with the toothed wheel to rotate the toothed wheel around its axis.

9. The system of claim 7, wherein the second portion (28) is pivotally assembled on the mobile ring (26) along an axis (Γ) substantially tangent to the patient's body, a toothed wheel sector (96) being attached to the mobile ring, the second portion comprising an endless screw (98) cooperating with the toothed wheel sector, the shaft (50) of the second drive system (34) rotating the endless screw.

10. The system of claim 7, wherein the means for supporting the device comprise a third portion (30) slidably assembled on the second portion (28) and connected to the device, the system comprising a third system (36) for driving the third portion with respect to the second portion.
